(19) United States
(12) Patent Application Publication
(10) Pub. No.: US 2014/0113912 A1
(43) Pub. Date: Apr. 24, 2014

(54) TREATMENT AND MANAGEMENT OF CNS DISORDERS

(71) Applicants: Antony D. Loebel, Larchmont, NY (US); Robert M. Silva, Livingston, NJ (US)

(72) Inventors: Antony D. Loebel, Larchmont, NY (US); Robert M. Silva, Livingston, NJ (US)

(73) Assignee: Dainippon Sumitomo Pharma Co., Ltd., Osaka (JP)

(21) Appl. No.: 14/116,396

(22) PCT Filed: May 11, 2012
(86) PCT No.: PCT/US2012/037447

§ 371 Date: Jan. 10, 2014

Related U.S. Application Data
(60) Provisional application No. 61/485,765, filed on May 13, 2011.

(51) Int. Cl. A61K 31/496; G01N 33/483

(52) U.S. Cl. CPC A61K 31/496; G01N 33/483
USPC 514/254.04; 73/866

(57) ABSTRACT

The present disclosure relates to methods of treating certain CNS disorders. The present disclosure also relates to biomarkers for monitoring or predicting the efficacy of a treatment for a CNS disorder by lurasidone, or a pharmaceutically acceptable salt, solvate, clathrate or stereoisomer thereof.

### 1. FIELD

Provided herein is methods of treating, preventing and/or managing certain CNS disorders. Also provided herein is monitoring of specific biomarkers in samples obtained from patients before and during therapy for CNS disorders. Also provided herein is monitoring of expression of one or more specific biomarkers for the treatment of the CNS disorders using compounds provided herein before and during the therapy.

### 2. BACKGROUND

Lurasidone is a compound exhibiting a pharmacological activity as a psychotropic agent. Lurasidone has a chemical name (3aR,4S,7R,7aS)-2-{(1R,2R)-2-[4-(1,2-benzisothiazol-3-yl)piperazin-1-ylmethyl]cyclohexylmethyl}hexahydro-4,7-methano-2H-isoindole-1,3-dione], and has the following formula:

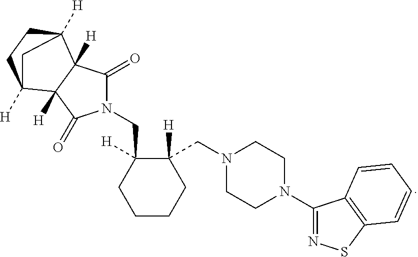 [SMILES: O=C1[C@@H]2[C@H]3CC[C@H](C3)[C@@H]2C(=O)N1C[C@@H]1CCCC[C@H]1CN1CCN(c2nsc3ccccc23)CC1]

Lurasidone is reported to have a high affinity for dopamine D₂, serotonin 5-HT_{1A}, 5-HT_{2A}, 5-HT₇, and noradrenaline α_{2C} receptors, moderate affinity for 5-HT_{1A} receptors, and minimal to no affinity for histamine H₁ and muscarinic M₁ receptors. Data from several placebo-controlled trials has demonstrated that lurasidone is effective in ameliorating the positive and negative symptoms of schizophrenia. Data from clinical and pre-clinical studies have suggested that lurasidone also demonstrates antidepressant- or anxiolytic-like effects, as well as pro-cognitive effects with potentially-reduced liability for extrapyramidal and CNS depressant side effects.

It has been reported that certain CNS disorders such as Alzheimer's disease are associated with low levels of glutamate. (See, e.g., Rupsingh et al., Neurobiology of Aging, 32: 802-810 (2011)). In addition, glutamate receptors, in particular NMDA receptors, have been implicated in various CNS disorders such as anxiety (Barkus et al., Eur. J Pharmacol., 626(1): 49-56 (2010)); borderline personality disorder (Grosjean et al., J Psychiatry Neurosci., 32(2): 103-115 (2007)); neuropathic pain (Parsons, Eur J. Pharmacol., 429: 71-78 (2001)); learning and memory impairment (Riedel et al., Behavioural Brain Res., 140:1-47 (2003)); schizophrenia (Patil et al 2007; Stahl, CNS Spectr., 12(4): 265-268 (2007)); and Alzheimer's disease (Farlow, Geriatrics, 59: 22-27 (2004)). Thus, modulation of glutamate and NMDA receptors can be an advantageous avenue for treating various CNS disorders.

While many treatments for CNS disorders have been contemplated, there still is an ongoing need for improved therapies. In certain cases, combination therapies or second-line therapies can play important roles in treating, preventing or managing CNS disorders. Further, a need exists for reliable biomarkers for the treatment of CNS disorders that can provide accurate assessment with regard to prognosis and efficacy of a particular treatment.

### 3. SUMMARY

Provided herein are methods of treating, preventing and/or managing a glutamate associated CNS disorder comprising administering to a patient a therapeutically or prophylactically effective amount of lurasidone, or a pharmaceutically acceptable salt, solvate, clathrate or stereoisomer thereof.

Also provided herein are methods of providing a second-line treatment for a CNS disorder provided herein comprising administering to a patient who had a prior therapy for the CNS disorder a therapeutically effective amount of lurasidone, or a pharmaceutically acceptable salt, solvate, clathrate or stereoisomer thereof.

Also provided herein are pharmaceutical compositions comprising lurasidone, or a pharmaceutically acceptable salt, solvate, clathrate or stereoisomer thereof, optionally in combination with one or more other therapeutic agents.

Also provided herein are dosing regimens for treatment with lurasidone, or a pharmaceutically acceptable salt, solvate, clathrate or stereoisomer thereof.

Provided herein are biomarkers for predicting or monitoring the efficacy of a treatment for a CNS disorder provided herein.

Also provided herein are methods for monitoring patient compliance with a drug treatment protocol.

### 4. DETAILED DESCRIPTION

### 4.1 BRIEF DESCRIPTION OF FIGURES

FIG. 1 illustrates improvement in PANSS total score, PANSS positive sub-scale and PANSS negative subscale scores in treatment responders after 6 weeks of treatment with lurasidone, olanzapine and placebo.

FIG. 2 illustrates glutamate levels in treatment responders at day 4 and week 6 after the initiation of the treatment with lurasidone, olanzapine and placebo.

FIG. 3 illustrates glutamate levels by PANSS negative subscore at week 6 following the initiation of treatment with lurasidone, olanzapine and placebo.

FIG. 4 illustrates a scatterplot that shows the correlation between change in the glutamate level and improvement in PANSS negative subscore at 6 weeks after the initiation of the treatment with lurasidone, olanzapine and placebo.

### 4.2 DEFINITIONS

As used herein, and unless otherwise specified, the terms “treat,” “treating” and “treatment” refer to an action that occurs while a patient is suffering from a CNS disorder provided herein, which reduces the severity of a CNS disorder provided herein, or retards or slows the symptoms associated therewith.

As used herein, unless otherwise specified, the terms “prevent,” “preventing” and “prevention” refer to the treatment with or administration of a compound provided herein prior to the onset of symptoms, particularly to patients at risk of a CNS disorder described herein. The term “prevention” includes the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

As used herein, and unless otherwise specified, the term “therapeutically effective amount” of a compound is an amount sufficient to provide a therapeutic benefit in the treatment or management of a CNS disorder provided herein, or to delay or minimize one or more symptoms associated with a CNS disorder provided herein. A therapeutically effective amount of a compound means an amount of therapeutic agent, alone or in combination with other therapies, which provides a therapeutic benefit in the treatment or management of a CNS disorder provided herein. The term “therapeutically effective amount” can encompass an amount that improves overall therapy, reduces or avoids symptoms or causes of a CNS disorder provided herein, or enhances the therapeutic efficacy of another therapeutic agent.

As used herein, and unless otherwise specified, a “prophylactically effective amount” of a compound is an amount sufficient to inhibit or reduce a symptom of a disease or to prevent recurrence of a disease. A prophylactically effective amount of a compound means an amount of therapeutic agent, alone or in combination with other agents, which provides a prophylactic benefit in the inhibition or reduction of a symptom of a disease or recurrence of a disease. The term “prophylactically effective amount” can encompass an amount that improves overall prophylaxis or enhances the prophylactic efficacy of another prophylactic agent.

As used herein, and unless otherwise specified, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucuronic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. In one embodiment, suitable are hydrochloric, hydrobromic, phosphoric, and sulfuric acids.

As used herein, and unless otherwise specified, the term “solvate” means a compound that further includes a stoichiometric or non-stoichiometric amount of solvent bound by non-covalent intermolecular forces. Where the solvent is water, the solvate is a hydrate.

As used herein, and unless otherwise specified, the term “stereoisomer” encompasses all enantiomerically/stereomerically pure and enantiomerically/stereomerically enriched compounds provided herein.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, greater than about 98% by weight of one stereoisomer of the compound and less than about 2% by weight of the other stereoisomers of the compound or greater than about 99% by weight of one stereoisomer of the compound and less than about 1% by weight of the other stereoisomers of the compound.

As used herein and unless otherwise indicated, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, and unless otherwise indicated, the term “enantiomerically pure” means a stereomerically pure composition of a compound having one chiral center. Similarly, the term “enantiomerically enriched” means a stereomerically enriched composition of a compound having one chiral center.

As used herein, and unless otherwise specified, the term “about,” when used in connection with a specific value, means that acceptable deviations from that value are also encompassed. In certain embodiments, the term “about” means that a value higher or lower than the given value by 1%, 3%, 5% 10%, 15%, 20%, 25%, 30%, 35% or 40% is encompassed.

The term “predict” generally means to determine or tell in advance. When used to “predict” the effectiveness of the treatment of a CNS disorder provided herein, for example, the term “predict” can mean that the likelihood of the outcome of the treatment can be determined at the outset, before the treatment has begun, or before the treatment period has progressed substantially.

The term “likelihood” generally refers to an increase in the probability of an event. The term “likelihood” when used in reference to the effectiveness of a patient response generally contemplates an increased probability that the symptoms of a CNS disorder provided herein will be lessened or decreased.

The term “monitor,” as used herein, generally refers to the overseeing, supervision, regulation, watching, tracking, or surveillance of an activity. For example, the term “monitoring the efficacy of a treatment for a CNS disorder” refers to tracking the effectiveness in treating a CNS disorder provided herein in a patient or in a sample, usually obtained from a patient. Similarly, the term “monitoring,” when used in connection with patient compliance, either individually, or in a clinical trial, refers to the tracking or confirming that the patient is actually following the treatment regimen being tested as prescribed.

As used herein, the terms that refer to any CNS disorders described herein elsewhere are used herein in a manner consistent with their accepted meanings in the art. See, e.g., Diagnostic and Statistical Manual of Mental Disorders, 4th Ed., American Psychiatric Association (1997) (DSM IV™).

As used herein, the term “modulator,” when used in connection with certain biochemicals or receptors, means an agent that can increase or decrease the level of the biochemicals or the activity of the receptors.

As used herein, the term “increase,” when referring to level or activity, means that the level or activity can be increased, for example, by about 5%, 10%, 20%, 30%, 40%, 50%, 60%, 70%, 90%, 100%, 200%, 300%, 500%, 1,000%, 5,000% or more of the comparative control level.

As used herein, the term “decrease,” when referring to level or activity, means that the level or activity can be decreased, for example, by about 99%, 95%, 90%, 80%, 70%, 60%, 50%, 40%, 30%, 20%, 10%, 1% or less of the comparative control level.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. Such samples can be, but are not limited to, organs, tissues, fractions, sera and cells isolated from a mammal (e.g., human). Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, PBMCs, tissue biopsies, and the like.

### 4.3 METHODS OF TREATMENT, PREVENTION AND/OR MANAGEMENT

Without being limited by a particular theory, based on metabolomic profiling of samples obtained from patients who had been treated with lurasidone, or a pharmaceutically acceptable salt, solvate, clathrate or stereoisomer thereof, an increase in the level of glutamate and related amino acid neurotransmitters was observed. Further without being limited by a particular theory, embodiments provided herein are based, in part, on the unexpected discovery that the administration of lurasidone, or a pharmaceutically acceptable salt, solvate, clathrate or stereoisomer thereof, results in an increase in the level of glutamate.

Accordingly, in certain embodiments, provided herein are methods of treating, preventing and/or managing a CNS disorder responsive to modulation of glutamate levels comprising administering to a patient a therapeutically or prophylactically effective amount of lurasidone, or a pharmaceutically acceptable salt, solvate (e.g., hydrate), clathrate or stereoisomer thereof.

Examples of CNS disorders include, but are not limited to, Alzheimer's disease, attention deficit disorder, attention deficit hyperactivity disorder, an anxiety disorder, bipolar disorder, borderline personality disorder, cognitive impairment associated with schizophrenia, learning and memory impairment, neuropathic pain, post-traumatic stress disorder, schizophrenia, negative symptoms associated with schizophrenia and schizoaffective disorder.

In one embodiment, the CNS disorder is not Alzheimer's disease, attention deficit hyperactivity disorder, bipolar disorder, schizophrenia, or negative symptoms associated with schizophrenia.

In another embodiment, the CNS disorder is Alzheimer's disease.

In another embodiment, the CNS disorder is attention deficit disorder.

In another embodiment, the CNS disorder is attention deficit hyperactivity disorder.

In another embodiment, the CNS disorder is an anxiety disorder.

Examples of anxiety disorders include, but are not limited to, general anxiety disorder, social anxiety disorder, panic disorder, posttraumatic stress disorder (PTSD) and obsessive-compulsive disorder.

In another embodiment, the CNS disorder is bipolar disorder.

Examples of bipolar disorders include, but are not limited to, bipolar I disorder, bipolar II disorder, and cyclothymic disorder.

In another embodiment, the CNS disorder is borderline personality disorder.

In another embodiment, the CNS disorder is learning and memory impairment or cognitive impairment associated with schizophrenia.

Examples of learning and memory impairment include, but are not limited to, decline in cognitive functions or cognitive domains, e.g., working memory, attention and vigilance, verbal learning and memory, visual learning and memory, reasoning and problem solving, e.g., executive function and/or speed of processing.

In another embodiment, the CNS disorder is neuropathic pain.

Examples of neuropathic pain include, but are not limited to, CRPS type I, CRPS type II, reflex sympathetic dystrophy (RSD), reflex neurovascular dystrophy, reflex dystrophy, sympathetically maintained pain syndrome, causalgia, Sudeck atrophy of bone, algoneurodystrophy, shoulder hand syndrome, post-traumatic dystrophy, trigeminal neuralgia, post herpetic neuralgia, cancer related pain, phantom limb pain, fibromyalgia, chronic fatigue syndrome, spinal cord injury pain, central post-stroke pain, radiculopathy, diabetic neuropathy, post-stroke pain, luetic neuropathy, and other painful neuropathic conditions such as those induced by drugs such as vincristine and velcade.

In another embodiment, the CNS disorder is schizophrenia or negative symptoms associated with schizophrenia.

Without being limited by a particular theory, it is believed that lurasidone, or a pharmaceutically acceptable salt, solvate, clathrate or stereoisomer thereof, can be effective as a second line therapy for a CNS disorder provided herein, in particular, where the first therapy did not involve modulation of glutamate level or NMDA receptors.

Thus, in some embodiments, provided herein are methods of treating, preventing and/or managing a CNS disorder responsive to modulation of glutamate levels, comprising administering to a patient who received a prior therapy a therapeutically or prophylactically effective amount of lurasidone, or a pharmaceutically acceptable salt, solvate (e.g., hydrate), clathrate or stereoisomer thereof.

In other embodiments, provided herein are methods of treating, preventing and/or managing a CNS disorder responsive to modulation of glutamate levels, comprising administering to a patient a therapeutically or prophylactically effective amount of lurasidone, or a pharmaceutically acceptable salt, solvate (e.g., hydrate), clathrate or stereoisomer thereof, in combination with one or more additional active agents.

Examples of CNS disorders are provided herein above.

In certain embodiments, the therapeutic agent used in the prior therapy, or the additional active agent, is a modulator of dopamine or serotonin receptors.

Examples of modulators of dopamine receptors include, but are not limited to, asenapine, iloperidone, paliperidone, blonanserin, perospirone, bromocriptine, carbergoline, pergolide, pramipexole, ropinirole, apomorphine, rotigotine, quinagolide, acepromazine, amisulpride, amoxapine, azaperone, benperidol, bromopride, butaclamol, clomipramine, chlorpromazine, chlorprothixene, clopenthixol, clozapine, domperidone, droperidol, eticlopride, flupenthixol, fluphenazine, fluspirilene, haloperidol, iodobenzamide, loxapine, mesoridazine, levomepromazine, metoclopramide, nafadotride, nemonapride, olanzapine, penfluridol, perazine, perphenazine, pimozide, prochlorperazine, promazine, quetiapine, raclopride, remoxipride, risperidone, spiperone, spiroxatrine, stepholidine, sulpiride, sultopride, tetrahydropalmatine, thiethylperazine, thioridazine, thiothixene, tiapride, trifluoperazine, trifluperidol, triflupromazine, ziprasidone, and combinations thereof.

Examples of modulators of serotonin receptors include, but are not limited to: azapirones such as buspirone, gepirone and tandospirone; triptans such as sumatriptan, rizatriptan and naratriptan; LY-334,370; lasmiditan; lorcaserin; cisapride; AS-19; katanserin; ondansetron; dolansetron; granisetron; quetiapine; methsergide; cyproheptapine; pizotifen; and combinations thereof.

Further without being limited by a particular theory, it was found that the modulation of glutamate levels by lurasidone correlates better with improvement in negative symptoms of schizophrenia (e.g., dimunition or loss of normal functions such as blunted affect, emotional withdrawal, poor rapport, passive/apathetic social withdrawal, difficulty in abstract thinking, lack of spontaneity and flow of conversation and stereotyped thinking) than with positive symptoms (e.g., excess or distortion of normal functions such as delusions, conceptual disorganization, hallucinations, hyperactivity, grandiosity, suspiciousness/persection and hostility).

Accordingly, in some embodiments, provided herein are methods of treating schizophrenia comprising administering to a patient, in whom negative symptoms of schizophrenia are dominant over other symptoms, a therapeutically effective amount of lurasidone, or a pharmaceutically acceptable salt, solvate, clathrate or stereoisomer thereof.

In other embodiments, provided herein are methods of treating schizophrenia in a patient who, after receiving a prior therapy, still suffers from persistent negative symptoms.

In certain embodiments, a pharmaceutically acceptable salt, solvate, clathrate or stereoisomer of lurasidone is used.

In one embodiment, a pharmaceutically acceptable salt of lurasidone is used. In another embodiment, a hydrochloride salt of lurasidone is used.

In one embodiment, a pharmaceutically acceptable solvate form of lurasidone, or salt thereof, is used. In another embodiment, the solvate is a hydrate.

In connection with all of the embodiments described above, any suitable route of administration can be employed for providing the patient with a therapeutically or prophylactically effective dose of an active ingredient.

The amount to be administered to a patient to treat, prevent, and/or manage the disorders described herein will depend upon a variety of factors including the activity of the particular compound employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount required. For example, the physician or veterinarian could start doses of the compounds employed at levels lower than that required in order to achieve the desired therapeutic effect and gradually increase the dosage until the desired effect is achieved.

In general, a suitable daily dose of a compound provided herein will be that amount of the compound which is the lowest dose effective to produce a therapeutic or prophylactic effect. Such an effective dose will generally depend upon the factors described above. The dosage may be formulated as a single or multiple unit dosage formulation. In one embodiment, the compound is given in single or divided doses per day.

In some embodiments, lurasidone, or a pharmaceutically acceptable salt, solvate, clathrate or stereoisomer thereof, may be used in an amount of from about 0.1 mg to about 500 mg per day, and can be adjusted in a conventional fashion (e.g., the same amount administered each day of the treatment, prevention or management period), in cycles (e.g., one week on, one week off), or in an amount that increases or decreases over the course of treatment, prevention, or management.

In other embodiments, the dose can be from about 1 mg to about 300 mg per day, from about 0.1 mg to about 160 mg per day, from about 1 mg to about 200 mg per day, from about 10 mg to about 120 mg per day, from about 20 mg to about 160 mg per day, from about 40 mg to about 120 mg per day, from about 10 mg to about 80 mg per day, from about 20 mg to about 80 mg per day, or from about 80 mg to about 160 mg per day. These doses can be administered in single or divided administrations.

In other embodiments, the dose can be about 10 mg per day, 20 mg per day, 30 mg per day, 40 mg per day, 50 mg per day, 60 mg per day, 70 mg per day, 80 mg per day, 90 mg per day, 100 mg per day, 110 mg per day, 120 mg per day, 130 mg per day, 140 mg per day, 150 mg per day, 160 mg per day, 170 mg per day, 180 mg per day, 190 mg per day, 200 mg per day or 240 mg per day. These doses can be administered in single or divided administrations.

In one embodiment, the dose is about 40 mg per day. In another embodiment, the dose is about 80 mg per day. In another embodiment, the dose is about 120 mg per day. In another embodiment, the dose is about 160 mg per day.

In some embodiments, lurasidone, or a pharmaceutically acceptable salt, solvate, clathrate or stereoisomer thereof, may be used in combination with one or more additional active agents to treat, prevent, and/or manage disorders described herein. In these embodiments, lurasidone, or a pharmaceutically acceptable salt, solvate, clathrate or stereoisomer thereof, can be administered simultaneously or sequentially with the additional active agent.

In one embodiment, the additional active agent is administered in an amount of from about 1 to about 1000 mg per day, from about 5 to about 500 mg per day, from about 10 to about 350 mg per day, or from about 50 to about 200 mg per day. The specific amount of the second active agent will depend on the specific agent used, the type of disease being treated or managed, the severity and stage of disease, and the amount(s) of compounds provided herein and any optional additional active agents concurrently administered to the patient.

### 4.4 PHARMACEUTICAL COMPOSITIONS AND DOSAGE FORMS

Pharmaceutical compositions can be used in the preparation of individual, single unit dosage forms. Pharmaceutical compositions and dosage forms provided herein comprise lurasidone, or a pharmaceutically acceptable salt, solvate, clathrate or stereoisomer thereof. Pharmaceutical compositions and dosage forms can further comprise one or more excipients.

Pharmaceutical compositions and dosage forms provided herein can also comprise one or more additional active ingredients. Examples of optional additional active ingredients are provided herein elsewhere.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intra-arterial), topical (e.g., eye drops or other ophthalmic preparations) administration to a patient. Examples of dosage forms include, but are not limited to: tablets; caplets; capsules, such as soft elastic gelatin capsules; cachets; troches; lozenges; suppositories; powders; aerosols (e.g., nasal sprays or inhalers); gels; liquid dosage forms suitable for oral or mucosal administration to a patient, including solutions, and elixirs; liquid dosage forms suitable for parenteral administration to a patient; and eye drops or other ophthalmic preparations suitable for topical administration.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th Ed., Mack Publishing, Easton Pa. (1990).

In one embodiment, pharmaceutical compositions and dosage forms comprise one or more excipients. Whether a particular excipient is suitable for incorporation into a pharmaceutical composition or dosage form depends on a variety of factors well known in the art including, but not limited to, the way in which the dosage form will be administered to a patient.

Also provided are pharmaceutical compositions and dosage forms that comprise one or more compounds that reduce the rate by which an active ingredient will decompose. Such compounds are referred to herein as “stabilizers”.

Like the amounts and types of excipients, the amounts and specific types of active ingredients in a dosage form may differ depending on factors such as, but not limited to, the route by which it is to be administered to patients.

In other embodiments, dosage forms comprise the second active ingredient. The specific amount of the second active agent will depend on the specific agent used, the diseases or disorders being treated or managed, and the amount(s) of a compound provided herein, and any optional additional active agents concurrently administered to the patient.

4.4.1 Oral Dosage Forms

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy. See generally, Remington's The Science and Practice of Pharmacy, 21st Ed., Lippincott Williams & Wilkins (2005).

Oral dosage forms provided herein are prepared by combining the active ingredients in an intimate admixture with at least one excipient. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Pharmaceutical compositions may be prepared by a conventional method by using a conventional pharmaceutically acceptable carrier which is usually used in the preparation of a conventional pharmaceutical formuation. Examples of excipients that can be used in oral dosage forms provided herein include, but are not limited to, binders, fillers, disintegrants, and lubricants. Examples of excipients include lactose, white sugar, glucose, starch, calcium carbonate, kaolin, talc, crystalline cellulose, silicic acid, etc.

Examples of binders suitable for use in the pharmaceutical compositions and dosage forms provided herein include, but are not limited to, water, ethanol, gelatin, carboxymethylcellulose, shellac, methylcellulose, gum arabic, tragacanth powder, polyvinylpyrrolidone.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation.

Disintegrants that can be used in pharmaceutical compositions and dosage forms include, but are not limited to, sodium arginate, agar powder, laminaran powder, sodium hydrogen carbonate, polyoxyethylenesorbitan fatty acid esters, sodium laurylsulfate, stearic acid monoglyceride.

Lubricants that can be used in pharmaceutical compositions and dosage forms include, but are not limited to, purified talc, stearate, boric acid powder, polyethylene glycol.

In one embodiment, a solid oral dosage form comprises a compound provided herein, and optional excipients, such as anhydrous lactose, microcrystalline cellulose, polyvinylpyrrolidone, stearic acid, colloidal anhydrous silica, and gelatin.

In one embodiment, the dosage form is an oral dosage form. In another embodiment, the oral dosage form is in the form of a capsule or a tablet.

In one embodiment, the oral dosage form is a composition described in U.S. Publication No. 2009/0143404, the entirety of which is incorporated herein by reference. Briefly, the oral dosage form comprises lurasidone, or a pharmaceutically acceptable salt, solvate, clathrate or stereoisomer thereof, and one or more excipients selected from pregelatinized starch, a water soluble excipient and a water soluble polymeric binder.

### 4.5 METHODS OF MONITORING THE TREATMENT USING BIOMARKERS

Provided herein are methods relating to the use of certain biochemicals as biomarkers to predict or ascertain the efficacy of a treatment for a CNS disorder provided herein.

A biological marker or “biomarker” is a substance whose detection indicates a particular biological state, such as, for example, the progress of a CNS disorder. In some embodiments, biomarkers can either be determined individually, or several biomarkers can be measured simultaneously.

In some embodiments, a “biomarker” indicates a change in the level of certain biomolecules that may correlate with the risk or progression of a disease, or with the susceptibility of the disease to a given treatment. In some embodiments, the biomarker is a neurotransmitter.

In some embodiments, the progress of treatment for a CNS disorder can be followed by monitoring the levels of certain biomolecules. Without being limited by a particular theory, it was found that upon treatment with lurasidone, or a pharmaceutically acceptable salt, solvate, clathrate or stereoisomer thereof, change in the levels of certain biomolecules are observed. Examples of such biomolecules include, but are not limited to, glutamate and certain amino acid neurotransmitters such as glycine, serine, aspartate, and glutamine.

Thus, in some embodiments, the invention relates to a method of assessing or monitoring patient response to treatment for a CNS disorder with lurasidone, or a pharmaceutically acceptable salt, solvate, clathrate or stereoisomer thereof. In certain such embodiments, a sample is obtained from the patient, and the levels of one or more of the above-described biomarkers are measured to determine whether their levels are increased or decreased compared to the levels prior to the initiation of the treatment.

In one embodiment, provided herein is a method of monitoring patient response to treatment for a CNS disorder with lurasidone, or a pharmaceutically acceptable salt, solvate, clathrate or stereoisomer thereof, comprising:
obtaining a first biological sample from the patient;
measuring the level of a marker selected from glutamate, glycine, serine, glutamine, aspartate and a combination thereof, in the first biological sample;
administering lurasidone, or a pharmaceutically acceptable salt, solvate, clathrate or stereoisomer thereof, to the patient;
thereafter obtaining a second biological sample from the patient;
measuring the level of the same marker in the second biological sample; and
comparing the levels of the marker obtained from first and second biological samples;
   wherein a changed level of the marker in the second biological sample indicates an effective response.

Examples of CNS disorders are provided herein elsewhere. In one embodiment, the CNS disorder is schizophrenia. In another embodiment, the CNS disorder is a negative symptom of schizophrenia. In another embodiment, the CNS disorder is a cognitive disorder. In another embodiment, the CNS disorder is Alzheimer's disease. In another embodiment, the CNS disorder is an anxiety disorder. In another embodiment, the CNS disorder is attention deficit disorder or attention deficit hyperactivity disorder.

In one embodiment, the change in the level of the biomarker is an increase. In another embodiment, the change in the level of the biomarker is a decrease.

In one embodiment, the level of glutamate is monitored. In certain embodiments, the increase in the level of glutamate is about 5%, 10%, 15%, 20%, 25% or 30% or more as compared to the level of glutamate in the first biological sample.

In another embodiment, the level of glycine is monitored. In certain embodiments, the increase in the level of glycine is about 5%, 10%, 15%, 20%, 25% or 30% or more as compared to the level of glycine in the first biological sample.

In another embodiment, the level of serine is monitored. In certain embodiments, the increase in the level of serine is about 5%, 10%, 15%, 20%, 25% or 30% or more as compared to the level of serine in the first biological sample.

In one embodiment, the level of glutamine is monitored. In certain embodiments, the decrease in the level of glutamine is about 5%, 10%, 15%, 20%, 25% or 30% or more as compared to the level of glutamine in the first biological sample.

In one embodiment, the level of aspartate is monitored. In certain embodiments, the change in the level of aspartate is about 5%, 10%, 15%, 20%, 25% or 30% or more as compared to the level of aspartate in the first biological sample.

In another embodiment, two or more of glutamate, glycine, glutamine, aspartate, and serine are monitored at the same time.

With regard to the administration of lurasidone, or a pharmaceutically acceptable salt, solvate, clathrate or stereoisomer thereof, any dosing regimen described herein elsewhere can be employed. In one embodiment, lurasidone, or a pharmaceutically acceptable salt, solvate, clathrate or stereoisomer thereof, can be administered at an amount of about 40 mg per day, in a single or divided doses. In another embodiment, lurasidone, or a pharmaceutically acceptable salt, solvate, clathrate or stereoisomer thereof, can be administered at an amount of about 80 mg per day, in a single or divided doses. In another embodiment, lurasidone, or a pharmaceutically acceptable salt, solvate, clathrate or stereoisomer thereof, can be administered at an amount of about 120 mg per day, in a single or divided doses. In another embodiment, lurasidone, or a pharmaceutically acceptable salt, solvate, clathrate or stereoisomer thereof, can be administered at an amount of about 160 mg per day, in a single or divided doses.

The second biological sample can be obtained at various time points after the initiation of the treatment with lurasidone, or a pharmaceutically acceptable salt, solvate, clathrate of stereoisomer thereof. In some embodiments, the second biological sample is obtained at 1 day, 2 days, 3 days, 4 days, 5 days, 6 days, 7 days, 10 days, 15 days, 3 weeks, 4 weeks, 5 weeks, 6 weeks, 2 months, 3 months, 4 months, 5 months or 6 months after the initiation of the treatment with lurasidone, or a pharmaceutically acceptable salt, solvate, clathrate or stereoisomer thereof.

In one embodiment, the second biological sample is obtained at 4 days after the initiation of the treatment with lurasidone, or a pharmaceutically acceptable salt, solvate, clathrate or stereoisomer thereof. In another embodiment, the second biological sample is obtained at 6 weeks after the initiation of the treatment with lurasidone, or a pharmaceutically acceptable salt, solvate, clathrate or stereoisomer thereof.

The biomarkers can also be used to track and adjust individual patient treatment effectiveness. The biomarkers can be used to gather information needed to make adjustments in a patient's treatment, increasing or decreasing the dose of an agent as needed. For example, a patient receiving a treatment compound can be tested using a biomarker to see if the dosage is becoming effective, or if a more aggressive treatment plan may be needed.

In other embodiments, these biomarkers can additionally be used to track or perform quality control on human research trials or to monitor patient compliance for a drug regimen by providing a means to confirm that the patient is receiving specific drug treatments. These biomarkers can be used in connection with, for example, the management of patient treatment, clinical trials, and cell-based research.

In one embodiment, these biomarkers can be used to track patient compliance during individual treatment regimes, or during clinical trials. For example, the levels of biomarkers can be followed at set intervals during a clinical trial to ensure that the patients included in the trial are taking the drugs as instructed. In addition, in the case of CNS disorders, where the patients' mental abilities are compromised, it is important to monitor the patients' compliance with the treatment protocol to ensure proper treatment is being administered.

The treatment of individual patients can also be followed using the biomarkers. For example, when the level of a particular biomarker is measured, an altered level of the biomarker compared to that of an untreated control indicates at least partial patient compliance with the drug treatment protocol.

Thus, in some embodiments, a method for assessing patient compliance with a drug treatment protocol is provided. A biological sample is obtained from the patient, and the levels of the biomarkers are measured and compared to that of a control untreated sample. An altered levels of biomarkers compared to those of an untreated control sample indicates compliance with the protocol.

In one embodiment, provided herein is a method for monitoring patient compliance with a treatment protocol for a CNS disorder with lurasidone, or a pharmaceutically acceptable salt, solvate, clathrate or stereoisomer thereof, comprising:
obtaining a first biological sample from the patient;
measuring the level of a biomolecule selected from glutamate, glycine, serine, glutamine, aspartate and a combination thereof, in the first biological sample;
initiating the patient with the treatment protocol;
obtaining a second biological sample from the patient; and
comparing the levels of the biomolecule in the first and second biological samples;
   wherein a changed level of the biomolecule in the second biological sample indicates patient compliance with the treatment protocol.

Examples of CNS disorders are provided herein elsewhere. In one embodiment, the CNS disorder is schizophrenia. In another embodiment, the CNS disorder is a negative symptom of schizophrenia. In another embodiment, the CNS disorder is a cognitive disorder. In another embodiment, the CNS disorder is Alzheimer's disease. In another embodiment, the CNS disorder is an anxiety disorder. In another embodiment, the CNS disorder is attention deficit disorder or attention deficit hyperactivity disorder.

In one embodiment, the level of glutamate is monitored. In certain embodiments, the increase in the level of glutamate is about 5%, 10%, 15%, 20%, 25% or 30% or more as compared to the level of glutamate in the first biological sample.

In another embodiment, the level of glycine is monitored. In certain embodiments, the increase in the level of glycine is about 5%, 10%, 15%, 20%, 25% or 30% or more as compared to the level of glycine in the first biological sample.

In another embodiment, the level of serine is monitored. In certain embodiments, the increase in the level of serine is about 5%, 10%, 15%, 20%, 25% or 30% or more as compared to the level of serine in the first biological sample.

In one embodiment, the level of glutamine is monitored. In certain embodiments, the decrease in the level of glutamine is about 5%, 10%, 15%, 20%, 25% or 30% or more as compared to the level of glutamine in the first biological sample.

In one embodiment, the level of aspartate is monitored. In certain embodiments, the change in the level of aspartate is about 5%, 10%, 15%, 20%, 25% or 30% or more as compared to the level of aspartate in the first biological sample.

In another embodiment, two or more of glutamate, glycine, glutamine, aspartate, and serine are monitored at the same time.

With regard to the administration of lurasidone, or a pharmaceutically acceptable salt, solvate, clathrate or stereoisomer thereof, any dosing regimen described herein elsewhere can be employed. In one embodiment, lurasidone, or a pharmaceutically acceptable salt, solvate, clathrate or stereoisomer thereof, can be administered at an amount of about 40 mg per day, in a single or divided doses. In another embodiment, lurasidone, or a pharmaceutically acceptable salt, solvate, clathrate or stereoisomer thereof, can be administered at an amount of about 80 mg per day, in a single or divided doses. In another embodiment, lurasidone, or a pharmaceutically acceptable salt, solvate, clathrate or stereoisomer thereof, can be administered at an amount of about 120 mg per day, in a single or divided doses. In another embodiment, lurasidone, or a pharmaceutically acceptable salt, solvate, clathrate or stereoisomer thereof, can be administered at an amount of about 160 mg per day, in a single or divided doses.

The second biological sample can be obtained at various time points after the initiation of the treatment with lurasidone, or a pharmaceutically acceptable salt, solvate, clathrate of stereoisomer thereof. In some embodiments, the second biological sample is obtained at 1 day, 2 days, 3 days, 4 days, 5 days, 6 days, 7 days, 10 days, 15 days, 3 weeks, 4 weeks, 5 weeks, 6 weeks, 2 months, 3 months, 4 months, 5 months or 6 months after the initiation of the treatment with lurasidone, or a pharmaceutically acceptable salt, solvate, clathrate or stereoisomer thereof.

In one embodiment, the second biological sample is obtained at 4 days after the initiation of the treatment with lurasidone, or a pharmaceutically acceptable salt, solvate, clathrate or stereoisomer thereof. In another embodiment, the second biological sample is obtained at 6 weeks after the initiation of the treatment with lurasidone, or a pharmaceutically acceptable salt, solvate, clathrate or stereoisomer thereof.

In certain embodiments, based, in part, on the finding that detectable increase or decrease in certain biomarkers are observed during the treatment of a CNS disorder by lurasidone, or a pharmaceutically acceptable salt, solvate, clathrate or stereoisomer thereof, the levels of these biomarkers may be used as an indicator for predicting the likelihood of responsiveness of a particular patient. Without being limited by a particular theory, as provided above, it is also known that low levels of glutamate are associated with patients suffering from certain CNS disorders.

Accordingly, determining the levels of biomarkers such as glutamate, glycine, glutamine, aspartate, and serine in patients suffering from a CNS disorder as described herein can provide useful information as to whether the patients would be responsive to the treatment with lurasidone, or a pharmaceutically acceptable salt, solvate, clathrate of stereoisomer thereof. In some embodiments, the level of the biomarker is measured in a biological sample obtained from a patient and compared with the level of the same biomarker in a non-patient.

In one embodiment, provided herein is a method of predicting whether a patient will be responsive to treatment for a CNS disorder with lurasidone, or a pharmaceutically acceptable salt, solvate, clathrate or stereoisomer thereof, comprising:
obtaining serum sample from the patient;
measuring the level of a biomarker selected from glutamate, glycine, glutamine, aspartate, serine and a combination thereof in the serum; and
comparing the level of the biomarker in the serum to that obtained from a non-patient;
   wherein a changed level of the biomarker indicates the likelihood of an effective patient response to the treatment with lurasidone.

Examples of CNS disorders are provided herein elsewhere. In one embodiment, the CNS disorder is schizophrenia. In another embodiment, the CNS disorder is a negative symptom of schizophrenia. In another embodiment, the CNS disorder is a cognitive disorder. In another embodiment, the CNS disorder is Alzheimer's disease. In another embodiment, the CNS disorder is an anxiety disorder. In another embodiment, the CNS disorder is attention deficit disorder or attention deficit hyperactivity disorder.

In one embodiment, the level of glutamate is monitored. In certain embodiments, the level of glutamate in the patient is about 5%, 10%, 15%, 20%, 25% or 30% or more lower as compared to the level of glutamate in the sample from non-patient.

In another embodiment, the level of glycine is monitored. In certain embodiments, the level of glycine in the patient is about 5%, 10%, 15%, 20%, 25% or 30% or more lower as compared to the level of glycine in the sample from non-patient.

In another embodiment, the level of serine is monitored. In certain embodiments, the level of serine in the patient is about 5%, 10%, 15%, 20%, 25% or 30% or more lower as compared to the level of serine in the sample from non-patient.

In one embodiment, the level of glutamine is monitored. In certain embodiments, the level of glutamine in the patient is about 5%, 10%, 15%, 20%, 25% or 30% or more higher as compared to the level of glutamine in the sample from non-patient.

In one embodiment, the level of aspartate is monitored. In certain embodiments, the level of aspartate is about 5%, 10%, 15%, 20%, 25% or 30% or more lower or higher as compared to the level of aspartate in the sample from non-patient.

In another embodiment, two or more of glutamate, glycine, glutamine, aspartate, and serine are monitored at the same time.

In one embodiment, provided herein is a method of improving negative symptoms of schizophrenia comprising administering to a patient a therapeutically effective amount of lurasidone, or a pharmaceutically acceptable salt thereof, wherein the likelihood of an effective patient response is predicted by a predicting method comprising:
obtaining a serum sample from the patient;
measuring the level of a biomarker selected from glutamate, glycine, serine, glutamine, aspartate and a combination thereof in the serum; and
comparing the level of the biomarker in the serum to that obtained from a non-patient;

wherein a changed level of the biomarker indicates the likelihood of an effective patient response.

In one embodiment, the level of glutamate is measured. In certain embodiments, the level of glutamate in the patient is about 5%, 10%, 15%, 20%, 25% or 30% or more lower as compared to the level of glutamate in the sample from non-patient.

In another embodiment, the level of glycine is measured. In certain embodiments, the level of glycine in the patient is about 5%, 10%, 15%, 20%, 25% or 30% or more lower as compared to the level of glycine in the sample from non-patient.

In another embodiment, the level of serine is measured. In certain embodiments, the level of serine in the patient is about 5%, 10%, 15%, 20%, 25% or 30% or more lower as compared to the level of serine in the sample from non-patient.

In one embodiment, the level of glutamine is measured. In certain embodiments, the level of glutamine in the patient is about 5%, 10%, 15%, 20%, 25% or 30% or more higher as compared to the level of glutamine in the sample from non-patient.

In one embodiment, the level of aspartate is measured. In certain embodiments, the level of aspartate is about 5%, 10%, 15%, 20%, 25% or 30% or more lower or higher as compared to the level of aspartate in the sample from non-patient.

In another embodiment, two or more of glutamate, glycine, glutamine, aspartate, and serine are monitored at the same time.

In one embodiment, provided herein is a method of improving negative symptoms of schizophrenia comprising administering to a patient a therapeutically effective amount of lurasidone, or a pharmaceutically acceptable salt thereof, wherein the likelihood of an effective patient is predicted by a predicting method comprising:
obtaining a first biological sample from the patient before lurasidone treatment;
measuring the level of a biomarker selected from glutamate, glycine, serine, glutamine, aspartate and a combination thereof in the first biological sample;
administering lurasidone, or a pharmaceutically acceptable salt thereof, to the patient;
thereafter obtaining a second biological sample from the patient;
measuring the level of the biomarker in the second biological sample; and
comparing the levels of the biomarker obtained from first and second biological samples;

wherein a changed level of the biomarker in the second biological sample indicates an effective response.

In one embodiment, the change in the level of the biomarker is an increase. In another embodiment, the change in the level of the biomarker is a decrease.

In one embodiment, the level of glutamate is measured. In certain embodiments, the increase in the level of glutamate is about 5%, 10%, 15%, 20%, 25% or 30% or more as compared to the level of glutamate in the first biological sample.

In another embodiment, the level of glycine is measured. In certain embodiments, the increase in the level of glycine is about 5%, 10%, 15%, 20%, 25% or 30% or more as compared to the level of glycine in the first biological sample.

In another embodiment, the level of serine is measured. In certain embodiments, the increase in the level of serine is about 5%, 10%, 15%, 20%, 25% or 30% or more as compared to the level of serine in the first biological sample.

In one embodiment, the level of glutamine is measured. In certain embodiments, the decrease in the level of glutamine is about 5%, 10%, 15%, 20%, 25% or 30% or more as compared to the level of glutamine in the first biological sample.

In one embodiment, the level of aspartate is measured. In certain embodiments, the change in the level of aspartate is about 5%, 10%, 15%, 20%, 25% or 30% or more as compared to the level of aspartate in the first biological sample.

In another embodiment, two or more of glutamate, glycine, glutamine, aspartate, and serine are monitored at the same time.

With regard to the administration of lurasidone, or a pharmaceutically acceptable salt, solvate, clathrate or stereoisomer thereof, any dosing regimen described herein elsewhere can be employed. In one embodiment, lurasidone, or a pharmaceutically acceptable salt, solvate, clathrate or stereoisomer thereof, can be administered at an amount of about 40 mg per day, in a single or divided doses. In another embodiment, lurasidone, or a pharmaceutically acceptable salt, solvate, clathrate or stereoisomer thereof, can be administered at an amount of about 80 mg per day, in a single or divided doses. In another embodiment, lurasidone, or a pharmaceutically acceptable salt, solvate, clathrate or stereoisomer thereof, can be administered at an amount of about 120 mg per day, in a single or divided doses. In another embodiment, lurasidone, or a pharmaceutically acceptable salt, solvate, clathrate or stereoisomer thereof, can be administered at an amount of about 160 mg per day, in a single or divided doses.

The second biological sample can be obtained at various time points after the initial administration of lurasidone, or a pharmaceutically acceptable salt, solvate, clathrate of stereoisomer thereof. In some embodiments, the second biological sample is obtained at about 1 day, 2 days, 3 days, 4 days, 5 days, 6 days, 7 days, 10 days, 15 days, 3 weeks, 4 weeks, 5 weeks, 6 weeks, 2 months, or 3 months after the initiation of the treatment with lurasidone, or a pharmaceutically acceptable salt, solvate, clathrate or stereoisomer thereof.

In one embodiment, the second biological sample is obtained about 4 days after the initial administration of lurasidone, or a pharmaceutically acceptable salt thereof.

Any conventional methods known in the art can be used to determine the levels of the above described biomarkers. In one embodiment, the levels of the biomarkers are determined using metabolomic analysis, an exemplary procedure is described in detail herein below. Briefly, samples are extracted and split into equal parts for analysis on the GC/MS and LC/MS/MS platforms. Proprietary software is used to match ions to a library of standards for metabolite identification and for metabolite quantitation by peak area integration.

Certain embodiments provided herein can be illustrated by the following Examples, which are not intended to limit the full extent of disclosure provided herein in any ways.

### 5. EXAMPLES

### 5.1 PANSS Scoring

Positive and Negative Syndrome Scale (PANSS) was measured according to the procedures well-established in the art. Clinical interviews were conducted by qualified trained professionals. A patient diagnosed schizophrenia was rated from 1 to 7 on 30 different symptoms based in the interview. The 30 symptoms assessed were: (1) positive symptoms, i.e., delusions, conceptual disorganization, hallucinations, hyperactivity, grandiosity, suspiciousness/persecution and hostility; (2) negative symptoms, i.e., blunted affect, emotional withdrawal, poor rapport, passive/apathetic social withdrawal, difficulty in abstract thinking, lack of spontaneity and flow of conversation and stereotyped thinking; and (3) general psychopathology symptoms, i.e., somatic concern, anxiety, guilt feelings, tension, mannerism and posturing, depression, motor retardation, uncooperativeness, unusual thought content, disorientation, poor attention, lack of judgment and insight, disturbance of volition, poor impulse control, preoccupation and active social avoidance. If a patient shows little or no symptom, the score for that symptom was rated 1, while a higher number was assigned for the symptom for higher severity.

PANSS assessments were conducted at Baseline and at each weekly visit during the 6-week double-blind treatment period. Baseline characteristics, including mean PANSS scores for the patient sample used for metabolomic analysis is shown below in Table 1.

TABLE 1
Baseline Characteristics of Patient Sample

| | | Lurasidone | Olanzapine | Placebo |
|---|---|---|---|---|
| | | (N = 40) | (N = 40) | (N = 40) |
| No. of Males (%) | | 33 (83) | 31 (78) | 27 (68) |
| Age mean (SD) | | 36.9 (11.6) | 37.6 (10.2) | 36.1 (11.0) |
| PANSS | Total | 95.9 (11.3) | 95.0 (10.8) | 94.2 (9.2) |
| mean | Positive | 25.9 (4.1) | 25.6 (4.0) | 25.4 (3.2) |
| (SD) | Negative | 24.2 (5.7) | 24.0 (5.7) | 23.0 (4.8) |

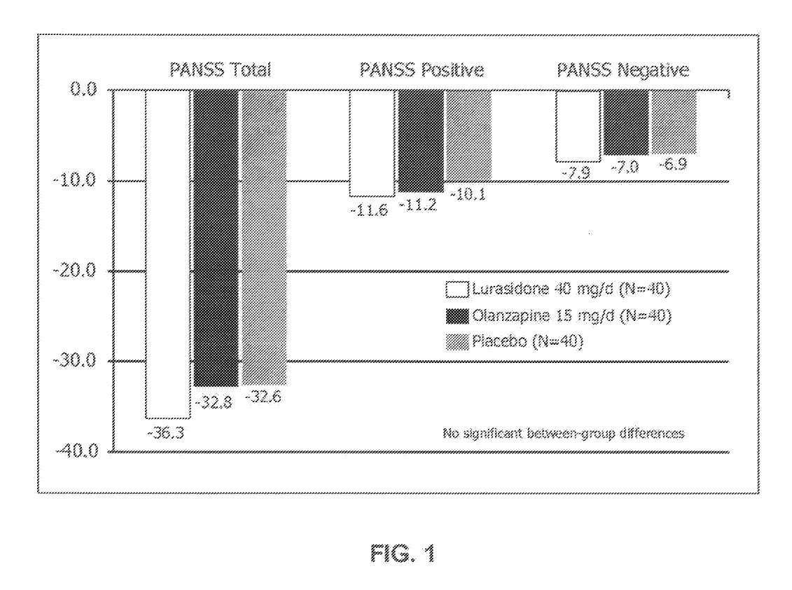

Prior to the baseline visit, subjects underwent a 3-7 day washout of their current antipsychotic medication and randomized to receive either lurasidone (40 mg per day), olanzapine (15 mg per day) or placebo for 6 weeks. Symptom improvement was assessed by the change in PANSS score (total and subscale scores) from baseline to study endpoint. A total of 40 subjects from each treatment group who demonstrated ≧30% improvement in PANSS total score (“treatment responders”) were selected for metabolomic analysis. Only subjects who completed the 6-week double-blind treatment period and who had adequate blood samples available (at both Day 4 and Week 6 timepoints) were considered for metabolomic analyis. As shown in FIG. 1, improvement in PANSS total as well as PANSS positive and negative subscores was comparable across the three treatment groups (lurasidone 40 mg/d; olanzapine 15 mg/d; or placebo).

### 5.2 Metabolomic Analysis

Blood samples were taken at 4 days and 6 weeks following treatment. Plasma samples were isolated from whole blood and used for global metabolomic profiling.

Sample Preparation:

At the time of analysis, samples were thawed and extracts prepared according to a standard protocol, which is designed to remove protein, dislodge small molecules bound to protein or physically trapped in the precipitated protein matrix, and recover a wide range of chemically diverse metabolites. A separate aliquot of each experimental plasma sample was taken then pooled for the creation of “Client Matrix” (CMTRX) samples. These CMTRX samples were injected throughout the platform run and served as technical replicates, allowing variability in the quantitation of all consistently detected biochemicals to be determined and overall process variability and platform performance to be monitored. Extracts of all experimental and CMTRX samples were split for analysis on the GC/MS and LC/MS/MS platforms.

Data Collection and Normalization:

The CMTRX technical replicate samples were treated independently throughout the process as if they were study samples. All process samples (CMTRX, GROBs—a mixture of organic components used to assess GC column performance, process blanks, etc.) were spaced evenly among the injections for each day and all client samples were randomly distributed throughout each day's run. Data were collected over eight platform run days and thus, ‘block normalized’ by calculating the median values for each run-day block for each individual compound. This minimizes any inter-day instrument gain or drift, but does not interfere with intra-day sample variability. Missing values (if any) were assumed to be below the level of detection for that biochemical with the instrumentation used and were imputed with the observed minimum for that particular biochemical.

Process Evaluation and Compound Summary:

A number of internal standards were added to each experimental and process standard sample just prior to injection into the mass spectrometers. A measure of the platform variability was determined by calculating the median relative standard deviation (RSD) for these internal standards. Table 2 below shows the median relative standard deviation (RSD) for the internal standards. Because these standards were added to the samples immediately prior to injection into the instrument, this value reflects instrument variation. In addition, the median relative standard deviation (RSD) for the biochemicals that were consistently measured in the CMTRX represents the total variability within the process for the actual experimental samples and the variability in quantitation of the endogenous metabolites within these samples (Table 2). Results for the CMTRX and internal standards indicated that the platform produced data that met process specifications.

TABLE 2
Quality Control Statistics

| Quality Control | |
|---|---|
| Sample (Matrix) | Median RSD |
| Internal Standards | 5% |
| Endogeneous | 10% |
| Biochemicals | |

Table 3 below shows the total number of metabolites detected in this study. This total corresponds to many biochemicals that matched a named structure in a reference library. The remaining represent distinct chemical entities, but no matching named biochemicals were found for the compounds.

TABLE 3
Number of Biochemicals Indentified
Compound Classification

| | |
|---|---|
| Total | 732 |
| Named/Identified | 382 |
| Unidentified | 350 |

Statistical Analysis:

Biochemical data were analyzed by ANOVA contrasts and a Two-way ANOVA.

T-Test Comparisons:

Two parameters are typically evaluated when considering statistical significance, namely the p-value and the q-value. The p-value relates the probability that two comparisons are the same; a low p-value (p≦0.05) is generally accepted as a significantly different result. The q-value describes the false discovery rate; a low q-value (q≦0.10) is an indication of high confidence in a result. Because of the multiple testing occurring in the data sets produced by metabolomic studies, data is often evaluated for false positives.

Statistical analyses of the data were performed on natural log-transformed data to reduce the effect of any potential outliers in the data. T-test comparisons were calculated using the one or both of the statistical software analysis programs: Array Studio (Omicsoft, Inc) or ‘R’ from the Free Software Foundation, Inc.

For t-test comparisons, within each group: placebo, lurasidone and olanzapine, the early versus the late time point was compared (i.e., 4 days versus 6 weeks). In addition, both lurasidone and olanzapine were compared with placebo at the appropriate time point and then lurasidone was compared with olanzapine at both 4 days and 6 weeks.

Pathway Statistic (Hotelling's T² Test):

Pathway statistic was analyzed using Hotelling's T² test, which is a multivariate version of the univariate two-sample t-test.

Repeated Measures ANOVA:

Repeated measures ANOVA was performed to leverage the data from the multiple time-points within the study. There were three tests embedded within the repeated measures ANOVA: 1) Group Main; 2) Time Main; and 3) Interaction (Group×Time). Essentially, the “Group” Main effect tests whether the means of the three groups are different when averaged across all time points. The Group main for this study was treatment (placebo versus lurasidone versus olanzapine). The “Time” Main effect examines whether the means at each time-point are different when averaged across the groups (i.e., 4 days versus 6 weeks). Finally, the “Interaction” asks whether the time profiles are non-parallel between the groups (non-parallel profiles signify a difference during the time-course between the groups).

Random Forest:

Random Forest is a supervised classification technique based on an ensemble of decision trees. For a given decision tree, a subset of samples was selected to build the tree, and then the remaining samples were predicted from this tree. This process was repeated thousands of times to produce a forest. The final classification was determined by computing the frequencies (“votes”) of predictions for each group over the whole forest.

To assess which variables contribute the most to the separation, an “importance” measure was computed using “Mean Decrease Accuracy.” This value was determined by randomly permuting a variable and then running the values through the trees and reassessing the prediction accuracy. If a variable is not important, then this procedure has little change in the accuracy (permuting random noise will give random noise), while if a variable is important, the accuracy drops after such a permutation.

Statistical Comparisons:

ANOVA contrasts were used to test pairs of means and Two-way ANOVAs were used to test for main effects and interactions between groups.

Results:

The number of biochemicals that were observed to be significantly different (p≦0.05, no q-value cut-off) by t-test analysis was determined, and the directional changes of these biochemicals and the estimated false discovery rate (FDR or q-value) for the subset of compounds with p≦0.05 were examined. A sizeable number of biochemicals were observed to be significantly different between the various treatment groups when compared to control and the FDR estimates for these comparisons were relatively low.

Specifically, it was found that, in the lurasidone group at Day 42, the serum levels of 25 of a total of 732 biochemicals (3%) were significantly changed (11 were significantly increased and 14 significantly decreased). In contrast, for olanzapine the serum levels of 100 biochemicals (14%) were significantly changed (54 were increased and 46 decreased).

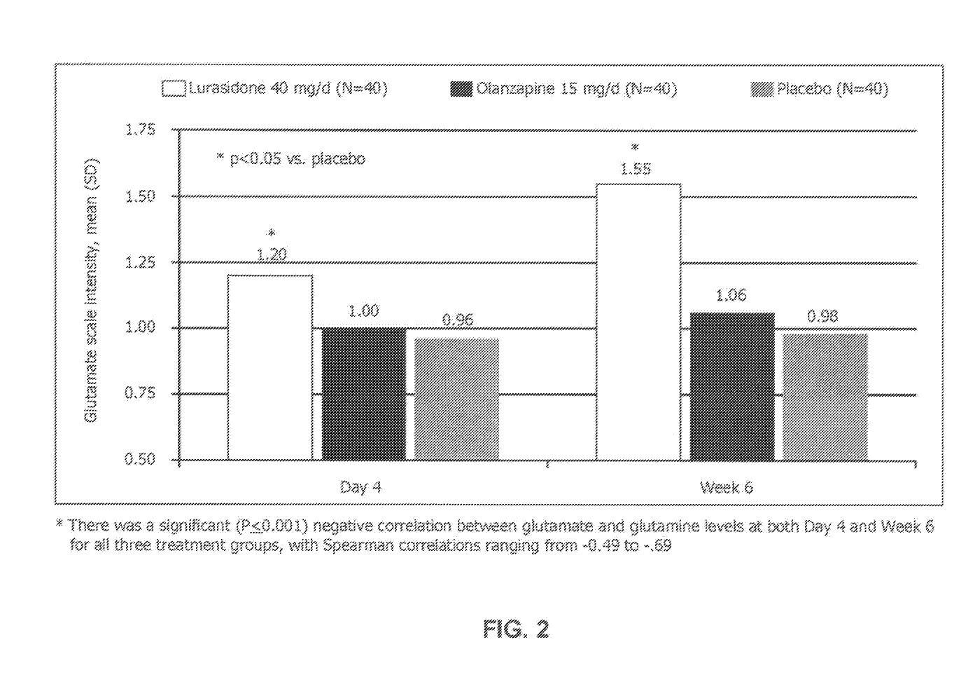

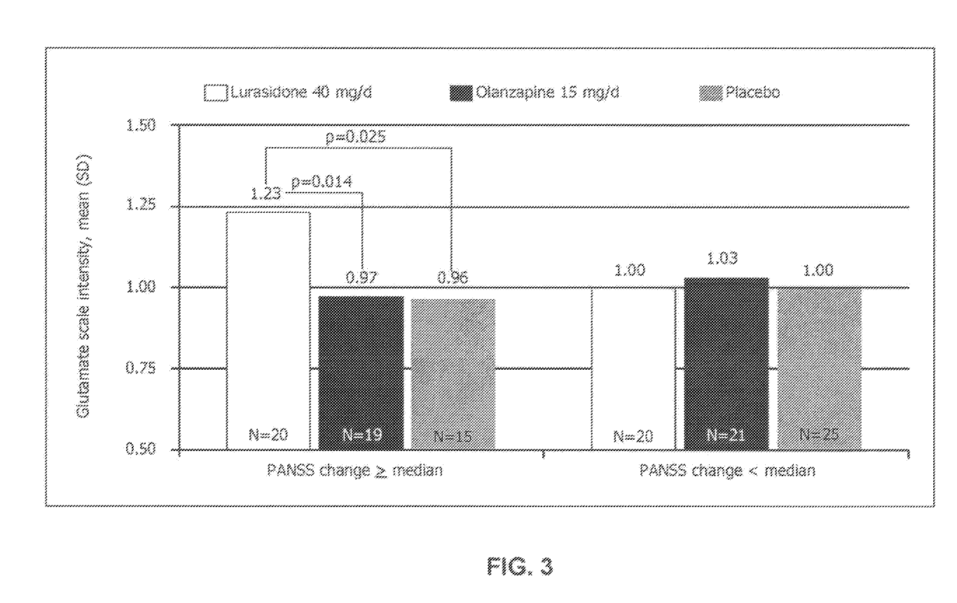

As noted above, FIG. 1 shows that each of the treatment groups (including only treatment responders) showed comparable efficacy in PANSS total, positive and negative subscores. However, as shown in FIG. 2, the overall glutamate level at days 4 and weeks 6 was significantly greater in the lurasidone treatment group than in the placebo group. No such effect was demonstrated for the olanzapine treatment group despite the efficacy comparable to the lurasidone group (FIG. 1). As shown in FIG. 3, lurasidone responders with high levels of improvement in negative symptoms had a significantly higher increase in glutamate levels compared to treatment responders in the olanzapine group or the placebo group suggesting that higher glutamate levels were associated with greater negative symptom improvement following treatment with lurasidone, but not olanzapine or placebo. In addition, it was found that the levels of certain amino acid neurotransmitters related to glutamate, such as glycine, serine and aspartate, also increased upon the treatment with lurasidone. The levels of glutamate for each of the treatment groups at week 6, each group further categorized into two subgroups (i.e., above and below median week 6 improvement), are illustrated in Table 4 below.

TABLE 4
Glutamate Levels at Week 6, mean (SD)

| | | Lurasidone | | Olanzapine | | Placebo | |
|---|---|---|---|---|---|---|---|
| Clinical Subgroups | | N | Glutamate | N | Glutamate | N | Glutamate |
| PANSS | Total Sample | 40 | 1.12 (0.34) | 40 | 1.00 (0.24) | 40 | 0.98 (0.40) |
| Total | >median change score | 23 | 1.13 (0.38) | 19 | 1.01 (0.27) | 16 | 1.07 (0.45) |
| Change at | ≦median change score | 17 | 1.09 (0.27) | 21 | 0.99 (0.26) | 24 | 0.93 (0.35) |
| Week 6 | | | | | | | |
| PANSS | Total Sample | 40 | 1.12 (0.34) | 40 | 1.00 (0.24) | 40 | 0.98 (0.40) |
| Negative | >median change score | 20 | 1.23* (0.36) | 19 | 0.97 (0.26) | 15 | 0.96 (0.29) |
| Change at | | | | | | | |
| Week 6 | ≦median change score | 20 | 1.00 (0.28) | 21 | 1.03 (0.23) | 25 | 1.00 (0.46) |
| PANSS | Total Sample | 40 | 1.12 (0.34) | 40 | 1.00 (0.24) | 40 | 0.98 (0.40) |
| Positive | >median change score | 22 | 1.14 (0.41) | 19 | 1.02 (0.27) | 19 | 1.09 (0.44) |
| Change at | ≦median change score | 18 | 1.09 (0.23) | 21 | 0.98 (0.22) | 21 | 0.89 (0.35) |
| Week 6 | | | | | | | |

*lurasidone vs. olanzapine: P = 0.014; lurasidone vs placebo: P = 0.025

As can be seen above, the level of glutamate among the subgroup, who were treated with lurasidone and showed better improvement in PANSS negative change score than median change, was observed to be significantly higher than all other responder groups. This result indicates that there likely is an association between glutamate levels and improvement in negative symptoms of schizophrenia. Moreover, a trend in favor of association of higher glutamate levels in lurasidone treatment group with greater improvements in total and positive subscores as compared to olanzapine and placebo groups was also observed.

According to the glutamate levels at the early stage of lurasidone treatment, for example, at 1 day, 2 days, 3 days, 4 days, 5 days, 6 days or 7 days after the initiation of the treatment with lurasidone, or a pharmaceutically acceptable salt, in each group further categorized into two subgroups (i.e., above and below median week 6 improvement in PANSS negative change score), the likelihood of an effective patient response to improve negative symptoms by lurasidone for each patient can be predicted.

### 5.3 Correlation Between Glutamate Levels and Change in PANSS Negative Subscore

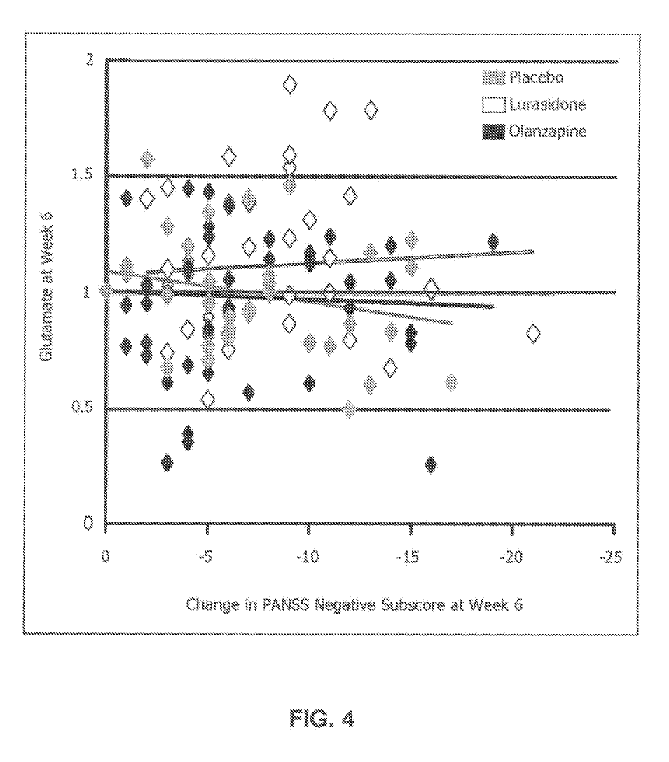

To assess whether there is any correlation between the levels of glutamate and changes in PANSS negative subscore, a scatterplot was generated and fitted. As shown in FIG. 4, it was observed that the levels of glutamate are proportional to the change in PANSS negative subscore at week 6 in patients who were treated by lurasidone. Neither olanzapine treated patients nor placebo treated patients showed such a pattern. This result indicates that an increase in the level of glutamate would likely have favorable effects in improving negative symptoms of schizophrenia.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A method of treating a CNS disorder responsive to modulation of glutamate levels, comprising administering to a patient a therapeutically effective amount of lurasidone or a pharmaceutically acceptable salt thereof, wherein the CNS disorder is an anxiety disorder, bipolar disorder, borderline personality disorder, learning and memory impairment or neuropathic pain.

2. The method of claim 1, wherein the lurasidone is in the form of a hydrochloride salt.

3. A method of treating a CNS disorder responsive to modulation of glutamate levels, comprising administering to a patient who received a prior therapy a therapeutically effective amount of lurasidone or a pharmaceutically acceptable salt thereof.

4. The method of claim 3, wherein the CNS disorder is Alzheimer's disease, an anxiety disorder, attention deficit disorder, attention deficit hyperactivity disorder, bipolar disorder, borderline personality disorder, learning and memory impairment, neuropathic pain or schizophrenia.

5. The method of claim 4, wherein the CNS disorder is schizophrenia.

6. The method of claim 3, wherein the lurasidone is in the form of a hydrochloride salt.

7. The method of claim 3, wherein the lurasidone or a pharmaceutically acceptable salt thereof is administered at a dose of about 40 mg, about 80 mg, about 120 mg or about 160 mg per day.

8. The method of claim 3, wherein the prior therapy is a treatment with a modulator of a dopamine receptor.

9. The method of claim 8, wherein the modulator of a dopamine receptor is bromocriptine, carbergoline, pergolide, pramipexole, ropinirole, apomorphine, rotigotine, quinagolide, acepromazine, amisulpride, amoxapine, azaperone, benperidol, bromopride, butaclamol, clomipramine, chlorpromazine, chlorprothixene, clopenthixol, clozapine, domperidone, droperidol, eticlopride, flupenthixol, fluphenazine, fluspirilene, haloperidol, iodobenzamide, loxapine, mesoridazine, levomepromazine, metoclopramide, nafadotride, nemonapride, olanzapine, penfluridol, perazine, perphenazine, pimozide, prochlorperazine, promazine, quetiapine, raclopride, remoxipride, risperidone, spiperone, spiroxatrine, stepholidine, sulpiride, sultopride, tetrahydropalmatine, thiethylperazine, thioridazine, thiothixene, tiapride, trifluoperazine, trifluperidol, triflupromazine, ziprasidone or a combination thereof.

10. The method of any claim 3, wherein the prior therapy is a treatment with a modulator of a serotonin receptor.

11. The method of claim 10, wherein the modulator of a serotonin receptor is buspirone, gepirone, tandospirone, sumatriptan, rizatriptan, naratriptan, LY-334,370, lasmiditan, lorcaserin, cisapride, AS-19, katanserin, ondansetron, dolansetron, granisetron, quetiapine, methsergide, cyproheptapine, pizotifen or a combination thereof.

12. The method of claim 3, wherein negative symptoms of schizophrenia persists after the prior treatment.

13-15. (canceled)

16. A method of treating schizophrenia comprising administering to a patient, in whom negative symptoms of schizophrenia are dominant over other symptoms, a therapeutically effective amount of lurasidone, or a pharmaceutically acceptable salt thereof.

17. The method of claim 16, wherein the lurasidone is in the form of a hydrochloride salt.

18. The method of claim 16, wherein the lurasidone or a pharmaceutically acceptable salt thereof is administered at a dose of about 40 mg, about 80 mg, about 120 mg or about 160 mg per day.

19. A method of monitoring patient response to treatment for a CNS disorder with lurasidone or a pharmaceutically acceptable salt thereof, comprising:
(a) obtaining a first biological sample from the patient;
(b) measuring the level of a marker selected from glutamate, glycine, serine, glutamine, aspartate and a combination thereof, in the first biological sample;
(c) administering lurasidone or a pharmaceutically acceptable salt thereof, to the patient;
(d) thereafter obtaining a second biological sample from the patient;
(e) measuring the level of the same marker in the second biological sample; and comparing the levels of the marker obtained from first and second biological samples;
wherein a changed level of the marker in the second biological sample indicates an effective response.

20. The method of claim 19, wherein the CNS disorder is Alzheimer's disease, an anxiety disorder, attention deficit disorder, attention deficit hyperactivity disorder, bipolar disorder, borderline personality disorder, learning and memory impairment, neuropathic pain or schizophrenia.

21. The method of claim 20, wherein the CNS disorder is schizophrenia.

22. The method of claim 19, wherein the level of glutamate obtained from the second biological sample is monitored, and an increase in glutamate level is about 5%, 10%, 15%, 20%, 25%, or 30% or more as compared to the level of glutamate in the first biological sample.

23. The method of claim 19, wherein the level of glutamine obtained from the second biological sample is monitored, and a decrease in glutamine level is about 5%, 10%, 15%, 20%, 25%, or 30% or more as compared to the level of glutamine in the first biological sample.

24. The method of claim 19, wherein the level of serine obtained from the second biological sample is monitored, and an increase in serine level is about 5%, 10%, 15%, 20%, 25%, or 30% or more as compared to the level of serine in the first biological sample.

25. The method of claim 19, wherein the lurasidone is in the form of a hydrochloride salt.

26. The method of claim 19, wherein the lurasidone or a pharmaceutically acceptable salt thereof is administered at a dose of about 40 mg, about 80 mg, about 120 mg or about 160 mg per day.

27. The method of claim 19, wherein the second biological sample is obtained about 4 days after the initial administration of lurasidone or a pharmaceutically acceptable salt thereof.

28. The method of claim 19, wherein the second biological sample is obtained about 6 weeks after the initial administration of lurasidone or a pharmaceutically acceptable salt thereof.

29-41. (canceled)

42. A method of improving negative symptoms of schizophrenia comprising administering to a patient a therapeutically effective amount of lurasidone or a pharmaceutically acceptable salt thereof, wherein the likelihood of an effective patient response is predicted by a predicting method comprising:
(a) obtaining a serum sample from the patient;
(b) measuring the level of a biomarker selected from glutamate, glycine, serine, glutamine, aspartate and a combination thereof in the serum; and
(c) comparing the level of the biomarker in the serum to that obtained from a non-patient;
wherein a changed level of the biomarker indicates the likelihood of an effective patient response; and
wherein the administration improves negative symptoms of schizophrenia.

43-45. (canceled)

46. A method of improving negative symptoms of schizophrenia comprising administering to a patient a therapeutically effective amount of lurasidone or a pharmaceutically acceptable salt thereof, wherein the likelihood of an effective patient response is predicted by a predicting method comprising:
(a) obtaining a first biological sample from the patient before lurasidone treatment; (b) measuring the level of glutamate in the first biological sample;
(c) administering lurasidone, or a pharmaceutically acceptable salt thereof, to the patient;
(d) obtaining a second biological sample from the patient;
(e) measuring the level of glutamate in the second biological sample; and comparing the levels of glutamate obtained from first and second biological samples;
wherein an increased level of the glutamate in the second biological sample indicates an effective response; and
wherein the administration improves negative symptoms of schizophrenia.

47-49. (canceled)

50. The method of claim 46, wherein the second biological sample is obtained about 1 day, about 2 days, about 3 days, about 4 days, about 5 days, about 6 days or about 7 days after the initial administration of lurasidone or a pharmaceutically acceptable salt thereof.

51. The method of claim 46, wherein the second biological sample is obtained about 4 days after the initial administration of lurasidone or a pharmaceutically acceptable salt thereof.
